(19) United States
(12) Patent Application Publication
(10) Pub. No.: US 2008/0108993 A1
(43) Pub. Date: May 8, 2008

(54) METHODS AND SYSTEMS FOR DEPLOYING SPINOUS PROCESS CONSTRAINTS

(75) Inventors: Ian Bennett, San Francisco, CA (US); Colin Cahill, San Francisco, CA (US); Todd Alamin, Woodside, CA (US); Louis Fielding, San Carlos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Simpirica Spine, Inc., Redwood City, CA (US)

(21) Appl. No.: 11/875,674

(22) Filed: Oct. 19, 2007

Related U.S. Application Data
(60) Provisional application No. 60/862,085, filed on Oct. 19, 2006.

(51) Int. Cl. A61B 17/58; A61B 17/08; A61B 17/32

(52) U.S. Cl. 606/61; 606/99; 606/151; 606/167

(57) ABSTRACT

Methods and kits for implanting constraints around spinous processes are described.

### CROSS-REFERENCES TO RELATED APPLICATIONS

This application claims the benefit of prior provisional application 60/862,085, (Attorney Docket No. 026398-000100), filed on Oct. 19, 2006, the full disclosure of which is incorporated herein by reference.

### BACKGROUND OF THE INVENTION

1. Field of the Invention

The present invention relates generally to medical methods and apparatus. More particularly, the present invention relates to methods and devices for restricting spinal flexion in patients having back pain or other spinal conditions.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine (FIG. 1). Pain tends to be exacerbated when patients put their lumbar spines into flexion (i.e. by sitting or bending forward) and relieved when they put their lumbar spines into extension (i.e. arching backwards). Discogenic pain can be quite disabling, and for some patients, can dramatically affect their ability to work and otherwise enjoy their lives.

This pain experienced by patients with discogenic low back pain can be thought of as flexion instability, and is related to flexion instability that is manifested in other conditions. The most prevalent of these is spondylolisthesis, a spinal condition in which abnormal segmental translation is exacerbated by segmental flexion. The device described here should as such also be useful for these other spinal disorders associated with segmental flexion, for which the prevention or control of spinal segmental flexion is desired.

Current treatment alternatives for patients diagnosed with chronic discogenic pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion is not usually recommended for discogenic pain because it is irreversible, costly, associated with high morbidity, and of questionable effectiveness. Despite its drawbacks, however, spinal fusion for discogenic pain remains common due to the lack of viable alternatives.

Recently, a less invasive and potentially more effective treatment for discogenic pain has been proposed. A spinal implant has been designed which inhibits spinal flexion while allowing substantially unrestricted spinal extension. The implant is placed over one or more adjacent pairs of spinal processes and provides an elastic restraint to the spreading apart of the spinal processes which occurs during flexion. Such devices and methods for their use are described in U.S. Patent Application 2005/02161017A1, published on Sep. 29, 2005, and having common inventors with the present application.

As illustrated in FIG. 2, an implant 10 as described in the '017 application, typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring of rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinal processes which provides a force that resists flexion. The force increases as the processes move further apart. Usually, the straps themselves will be essentially non-compliant so that the degree of elasticity or compliance may be controlled and provided solely by the compliance members 16.

The manner in which flexion is restricted with such an implant is controlled in part by the physical characteristics of the implant and in part by the way in which it is implanted in the patient. The physician controls the anterior-posterior location on the spinous processes at which the strap is placed. In a preferred embodiment, the physician can adjust the elasticity of the implant. In a preferred embodiment, the physician can further adjust the final size of the implant; in particular, the physician can adjust the effective length of the implant. The effective length of the implant is the length of the portion of the implant that is engaged when the patient flexes. If the implant is a continuous structure as in FIG. 2, the effective length is the inner perimeter of the structure. If the implant is a structure that passes around one spinous process and is attached at two ends to a spinous process or sacrum, as described in U.S. patent application Ser. No. 11/777,366, filed on Jul. 13, 2007, and having common inventors with the present application, the effective length is the distance along the tether structure between the attachment points.

The manner in which flexion is restricted with such an implant can have a significant effect on the surgical outcome for a patient. If an implant intended to restrict flexion is deployed such that it applies too much tension to the spinous processes, the patient may develop complications such as facet arthropathy or lateral recess stenosis. If an implant intended to restrict flexion applies too little tension to the spinous processes, it will have little impact when the segment undergoes a small amount of flexion from the natural neutral position and hence the patient's pain may not be adequately relieved.

This problem of balancing the need to deploy the implant such that it applies enough tension to the spinous processes to relieve pain but not so much tension that it causes complications is unique to this implant. Other structures deployed near or around the spinous processes such as those described by Bevan (U.S. Pat. No. 5,725,582) and Graf (US2004/0116927) are typically adjusted to be as tight as possible, either locking patients into extension or immobilizing the spinal segment in conjunction with a spinal fusion.

For these reasons, it would be desirable to provide methods and tools so that a physician can easily deploy the implant such that it applies an amount of tension on the spinous processes that is sufficient to relieve pain but not excessive. As such, the following invention relates to methods and tools for use in positioning and deploying an implant like that described in US2005/0216017A1.

2. Description of the Background Art

US 2005/0216017A1 has been described above. Other patents and published applications of interest include: U.S. Pat. Nos. 4,966,600; 5,011,494; 5,092,866; 5,116,340; 5,282,863; 5,395,374; 5,415,658; 5,415,661; 5,449,361; 5,456,722; 5,462,542; 5,496,318; 5,540,698; 5,609,634; 5,645,599; 5,725,582; 5,902,305; Re. 36,221; 5,928,232; 5,935,133; 5,964,769; 5,989,256; 6,053,921; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; Published U.S. Patent Application Nos. US 2002/0151978; US 2004/0024458; US 2004/0106995; US 2004/0116927; US 2004/0117017; US 2004/0127989; US 2004/0172132; US 2005/0033435; US 2005/0049708; US 2006/0069447; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; and Published Foreign Application Nos. EP 0322334 A1; and FR 2 681 525 A1.

### BRIEF SUMMARY OF THE INVENTION

The present invention provides methods and tools for the deployment of spinal implants for restricting flexion of spinal segments for the treatment of discogenic pain and other spinal conditions, such as spondylolisthesis, where a physician may desire to control segmental flexion. The methods comprise piercing an interspinous ligament with a tool to form a penetration above a first superior spinous process, advancing a first end of a tether through the penetration, piercing an interspinous ligament with a tool to form a second penetration below an inferior spinous process, advancing a second end of a tether through the second penetration, and joining the ends of the tether to form a continuous structure. The methods further comprise advancing separate first and second tethers through the penetrations and joining more than one pair of ends to form the continuous structure. Another aspect of the present invention may further include passing a guidewire along the path desired for the tether and using the guidewire to direct the tether into position around the spinous processes. In all aspects of the present invention, the steps described for positioning the implant preferably minimally disrupt the muscles, tendons, and ligaments so as to preserve intact as much of the native anatomy as possible. In particular the methods described will in all cases avoid disruption of the supraspinous ligament. Moreover, in most cases, the methods further comprise obtaining exposure to the preferred location for the implant without significantly disrupting the multifidus muscles.

In some patients, the geometry of the spinous process S1 on the sacrum may be such that the tether may not be adequately secured by passing it below the spinous process. In such instances, the methods may further comprise creating a hole in the sacrum and passing the tether structure through the hole, or inserting a sacral attachment member such as a hook or islet in the sacrum and passing the tether structure around or through the attachment member.

In another aspect of the present invention, the methods may further comprise treating a spinous process. Treating may consist of creating a depression in the spinous process in which the band can rest. Such a depression could be creating by any means of removing soft tissue and bone, including sanding, grinding, drilling, or notching. Treating may alternatively comprise delivering a chemical or biological preparation to the spinous process, such as a preparation of stem cells, growth factors, adhesives, or a chemical coating. Such a preparation may promote or prevent growth of the spinous process into or around the tether structure. The methods herein described for treating the spinous process may help to improve the biological interaction between the tether structure and the spinous process, such that potential complications such as inflammation, wear, and cracking are minimized.

In one embodiment of the present invention, the tether structures are alone joined to form a full continuous structure. A portion of the tether structures may provide an elastic resistance to elongation in response to an elongation force which results from flexion of the spinal segments between the adjacent spinous processes and/or the sacrum. Often, the tether structures will include at least two compliance members positioned such that they will lie symmetrically on opposite sides of the spinous processes when implanted.

In another embodiment of the present invention, additional components may be joined with the tether structure to form the continuous structure. Such components could be compliance members, tension members, compression members, adjustment members, or attachment members. Often, at least two compliance members are joined and positioned as part of the continuous structure such that they will lie symmetrically on opposite sides of the spinous processes when implanted. The compliance members will typically be coupled to non-compliant and/or cable components of the tether structure so that it is the compliance members which provide most or all of the compliance or elasticity in the implants.

In some cases, it will be desirable to deploy such an implant across a spinal segment which does not have a sufficient inferior spinous process for retaining a continuous structure. In these cases, the invention may further comprise providing an islet or hole in the lower vertebra or sacrum. Alternatively, rather than join the ends of the tethers to form a continuous structure, two separate ends which extend from a structure that is already passed above a superior spinous process may be anchored to the adjacent vertebra or sacrum using screws, dowels, staples, or any of the techniques described above.

In a further aspect of the present invention, the methods include using images of the patient's spine to determine the appropriate positioning and tensioning of the implant. Because the implant is designed to restrict flexion of the treated spinal segment, the physician may perform lateral radiographs in neutral, flexion, and extension positions. Of particular interest, the physician may note the segmental angles or spinous process distances at the segment to be treated. In one aspect of the present invention, the method includes a lateral radiograph to determine the distance between the points at which an implant would be likely to attach to the bone. For example, the method may include measuring the distance from the edge along the top of the superior spinous process where the structure would likely rest to the edge along the bottom of the inferior spinous process where the structure would likely rest in a lateral radiograph in the standing position. Such distance, or any other corresponding measurement, could then be subsequently used during the surgery to provide guidance for the physician with respect to positioning and tensioning of the implant.

Because the manner in which flexion is restricted can have a significant effect on the surgical outcome for a patient, the method further provides steps for determining an ideal position along the spinous process at which to deploy the tether. This may typically include determining the position above or below the spinous process at which to pierce the interspinous ligament to create the penetration through which the tether will be advanced. In one embodiment, the method includes engaging a positioning guide against a preselected anatomical landmark and positioning the tether along an axis provided by the guide. In a preferred embodiment, the method includes engaging the guide against the base of the spinous process or against the lamina near the base of the spinous process and positioning the tether along an anterior-posterior axis defined by the guide, although naturally such a positioning guide could be engaged with other anatomical landmarks. The positioning guide may be provided with a feature for engaging with the tool that penetrates the interpinous ligament. In an alternative embodiment, a single tool may be capable of both positioning the targeted penetration site relative to an anatomical landmark and creating the penetration. Such a joint tool may have one blunt aspect which engages with the base of the spinous process and extends along an anterior-posterior axis along the edge of the spinous process and a second sharp aspect which can be deployed perpendicular to the anterior-posterior axis to create the penetration at the targeted position in the interspinous ligament next to the spinous process.

Because the manner in which flexion is restricted can have a significant effect on the surgical outcome for a patient, in a further aspect of the present invention, the method includes adjusting the continuous structure such that the implant applies a desired amount of tension on the spinous processes.

In one embodiment, the method for adjusting the continuous structure includes changing the elasticity of the continuous structure. In a preferred embodiment, the elasticity change is effected by changing compliance components in the continuous structure. Stiffer compliance components may be included in the continuous structure for patients in need of greater flexion resistance, and less stiff compliance components may be joined in the continuous structure for patients in need of less flexion resistance. In another embodiment, the compliance component itself may be intentionally pre-tensioned or pre-relaxed in order to change the elasticity of the continuous structure.

In a more typical embodiment, the method for adjusting the implant includes changing the effective length of the structure. For embodiments that are continuous loops, the effective length of the structure is typically the inner perimeter of the continuous loop. Although the discussion here and will focus on changes to the inner perimeter of a continuous loop, it is recognized that similar methods apply to changing the effective length of a tether structure that passes around one spinous process and is attached at two ends to a spinous process or sacrum, for which the effective length consists of the length of the structure from one fixed attachment point to the other.

The methods for changing the effective length of the structure comprise increasing or decreasing the length of the portion of the tether that is engaged when the patient flexes. For example, it may be desirable to decrease the effective length of the tether. Such decreases may be effected by removing a length of the tether from the continuous structure. Often, this is accomplished by changing to position at which components in the tether structure are attached to each other such that some portion of the tether structure which was previously engaged during flexion is subsequently outside of the inner perimeter of the structure. For example, if the continuous structure includes an attachment element that clamps one portion of the tether, the attachment could would be loosened, more of the tether could be passed through the attachment to the outside of the loop, and the attachment could be tightened again to reform a continuous loop with a reduced inner perimeter and thus an implant with a smaller effective length. Such decreases may also be effected by swapping in and out components of the tether structure.

In one aspect of the present invention, measurements from images of a patient's spine are used to identify the desired effective length of the tether structure. The tether structure effective length may thus be adjusted based on information from such images until the desired effective length is reached.

In one aspect of the present invention, the method includes selecting and adjusting the components of the implant outside of the body, such that the implant, when deployed and joined into a continuous structure, already consists of the desired effective length. In another aspect of the present invention, the tether is engaged with a fixture outside of the body and the effective length of the tether structure is adjusted on the fixture.

In another aspect of the present invention, the method provides for adjusting the tether structure during the surgery until the desired effective length is reached. The methods and tools described include features that could aid the physician in determining when the tether structure has been adjusted to the desired effective length. In one embodiment, the tether includes visual indicators of the length, which might be colored regions of the tether or marks on the tether or other components of the tether structure. Alternative components such as strain gauges or digital readouts in the implant or the tool could alternatively indicate the length or tension in the tether structure. In one aspect of the present invention, the indicators are visible on x-ray or MRI, allowing the physician to use imaging to intraoperatively determine the effective length when the patient is in multiple positions.

Of particular importance are methods and tools that help the surgeon avoid unwanted slack in the tether structure. Such slack can consist of extra material that the surgeon fails to account for and that causes the actual effective length to be greater than the desired effective length. In one aspect, the tools include a tensioning block that is temporarily placed between the spinous processes across the spinal segment, such that the tether structure can be tensioned against the spinous processes without the spinous processes moving into extension. The tensioning block is then removed once the tether is adjusted so that no implant remains between the spinous processes. As an alternative to a block between the spinous processes, a tool could clamp the superior spinous process and clamp the inferior spinous process from both sides of the midline and then hold the clamps at a fixed distance from each other while the tether structure is tightened against the spinous processes. Other means for holding the spinous processes at a fixed distance from each other while the tether structure is tightened against them are also possible.

Systems according to the present invention include implants and tools. In one embodiment, such systems include at least one tether, a piercing tool having a tissue-penetrating distal tip and an anchor for releasably attaching an end of the tether; wherein the piercing tool is adapted to be advanced in an anterior direction toward the interspinous ligament and laterally so that the tissue-penetrating tip can be pierced through the ligament to push or pull the attached tether through the resulting penetration. Such systems may further include a tool for positioning the penetrations in the interspinous ligament at a targeted region along an anterior-posterior axis of a spinous process. Such systems often will further include an adjustment tool for adjusting the effective length of the tether structure. In addition, such systems often will further include stabilizing tools for maintaining the position of the implant and/or the spinous processes while the adjusting tool engages with the tether structure to adjust the effective length of the tether structure.

### BRIEF DESCRIPTION OF THE DRAWINGS

FIG. 1 is a schematic diagram illustrating the lumbar region of the spine including the spinous processes (SP), facet joints (FJ), lamina (L), transverse processes (TP), and sacrum (S).

FIG. 2 illustrates a spinal implant of the type described in US 2005/0216017A1.

FIGS. 3A-3D illustrate an exemplary embodiment of the method for delivering an implant consisting of two tether structures with attached compliance members joined to form a single tether structure and adjusted to apply the targeted amount of tension to the spinous processes.

FIG. 4 is illustrates an exemplary embodiment of a stabilizing tool that maintains the position of the spinous processes to prevent them from moving towards each other when tension is applied to the tether structure during the adjustment process.

FIG. 5 is an exemplary embodiment of an adjustment tool used to change the effective length of the band.

FIG. 6 is a schematic illustration of a positioning tool that is deployed along the spinous process to determine appropriate placement of the tether structure.

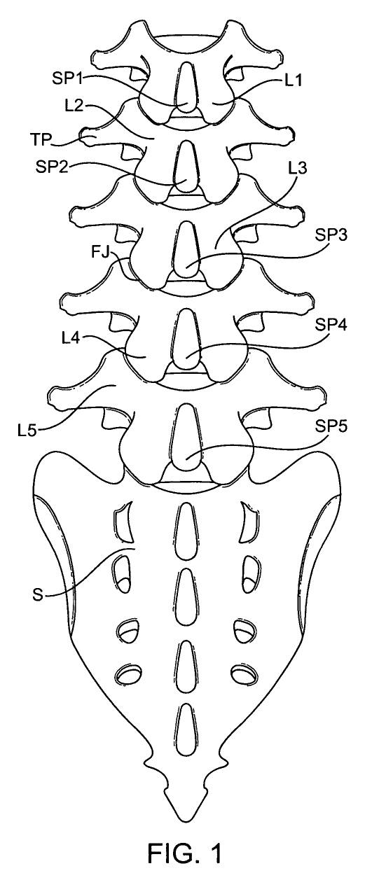

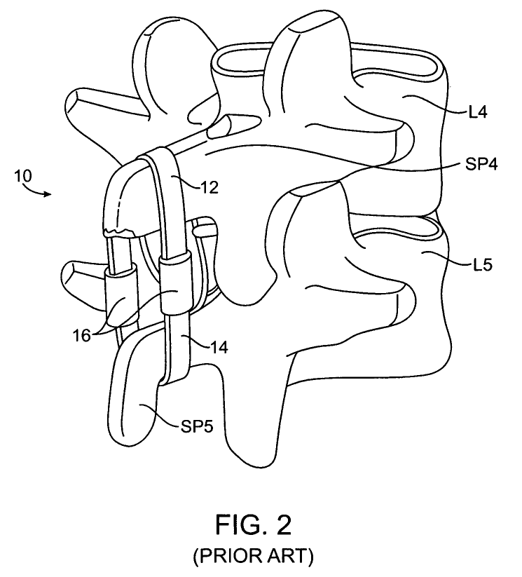

### DETAILED DESCRIPTION OF THE INVENTION

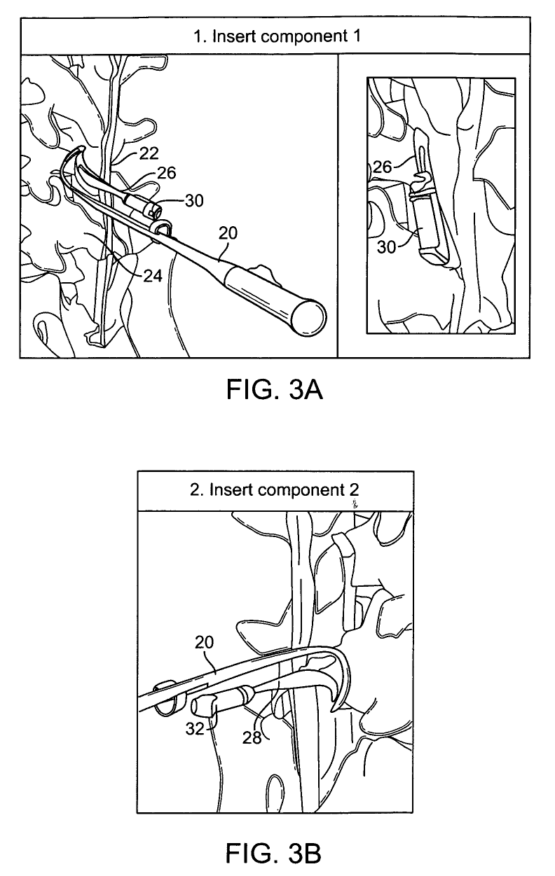

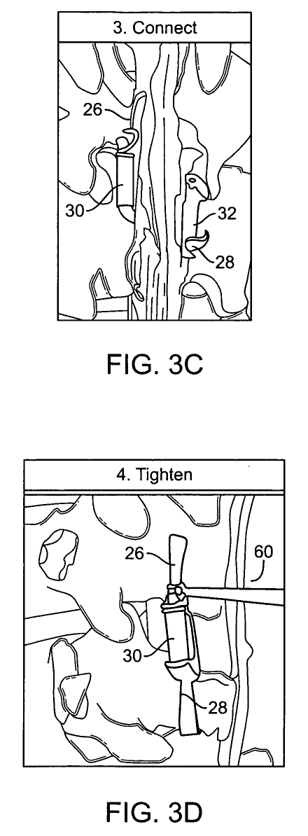

Referring now to FIGS. 3A-3D, a tool 20 suitable for use in accordance with the methods of the present invention is used to create penetrations above a superior spinous process 22 and below an inferior spinous process 24. Tethers 26 and 28 including pre-attached compliance members 30 (FIG. 3A) and 32 (FIG. 3B) are advanced through the penetrations and joined (FIG. 3C) to form a continuous, multi-component tether structure. Typically, the tether structure is then adjusted (FIG. 3D) to apply a desired amount of tension to the spinous processes, typically in the range from 0N to 30N, usually from 0N to 5N, assuming that the spinous processes are unconstrained during the tensioning process.

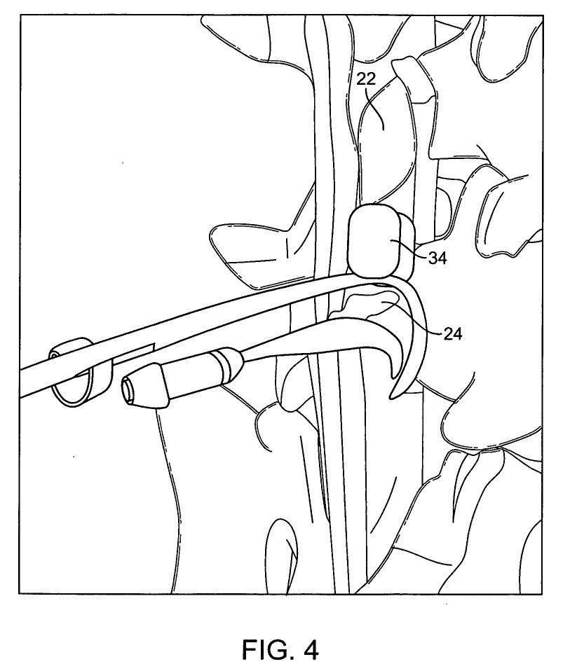

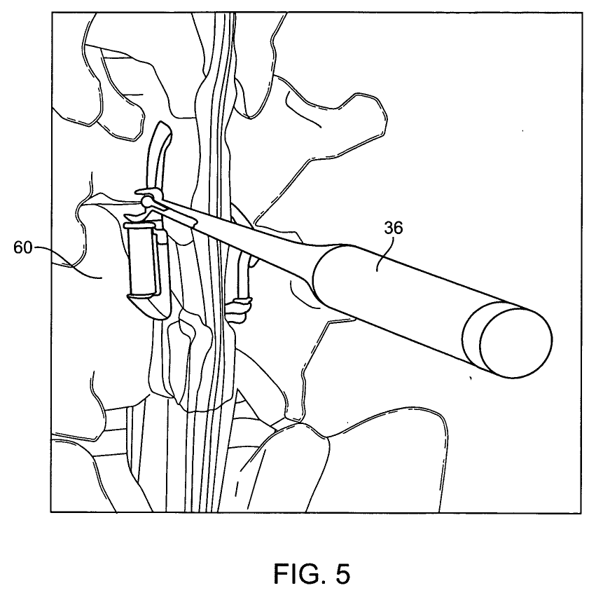

Referring now to FIG. 4, a tensioning block 34 or other stabilizing tool is optionally provided between the spinous processes to keep the spinous processes from extending while the tension on the tether structure is adjusted. Such stabilizing tools can allow the physician to remove unwanted slack from the deployed implant to achieve a targeted effective length and/or tension for the implant. When the tether structure is adjusted while such a stabilizing tool prevents the spinous processes from extending, the targeted tension applied to the spinous processes during the adjustment procedure may be higher than the ranges described above for the method in which no such stabilizing tool is provided.

Referring now to FIG. 5, the system and methods further comprise an adjustment tool 36 which engages with the tether structure 60 to change the effective length of the tether structure. Typically, an adjustment mechanism such as spool mechanism can be provided as part of the compliance members 30 and 32 to allow for tightening or loosening of the tethers 26 and 28.

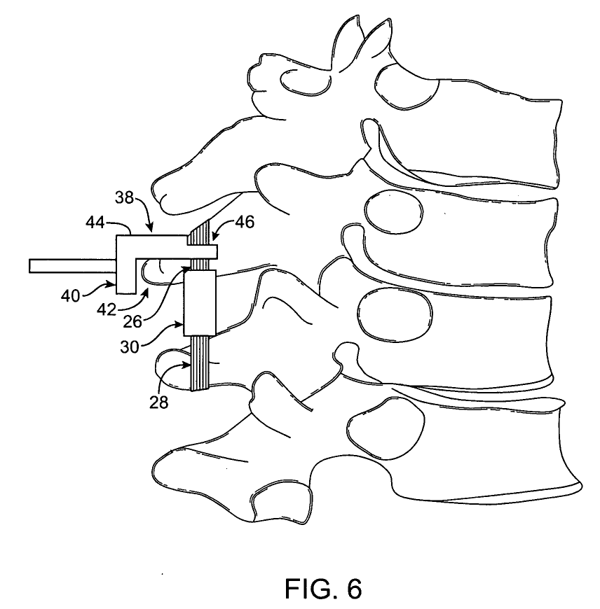

Referring now to FIG. 6, a positioning tool or jig 38 is optionally used to create an anterior-posterior axis to determine the target location at which to form the penetrations of the interspinous ligaments through which the position the tether structure will be advanced. The tool 38 has an end or stop 40 which engages the distal end 42 of the spinous process and a shaft or body 44 which defines a desired off set length and which has a location 46 for receiving and positioning the tool 20 and/or tether 26 of the implant.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for constraining spinous processes to elastically limit flexion of a spinal segment, said method comprising:
piercing an interspinous ligament with a tool to form a first penetration above an upper side of a first spinous process;
advancing a first end of a tether through the first penetration;
piercing the intraspinous ligament with the tool to form a second penetration below a lower side of a second spinous process;
advancing a second end of a tether through the second penetration; and joining at least the first and second tether ends to form a tether structure wherein the structure elastically couples an upper spinous process and a lower spinous process.

2. A method as in claim 1, wherein the second penetration is formed through the lower spinous process or sacrum.

3. A method as in claim 1, wherein advancing the first and second tether ends comprises attaching the tether ends to the tool and pushing the ends through the penetrations as they are formed.

4. A method as in claim 1, wherein advancing the first and second tether ends comprises attaching the tether ends to the tool after the penetrations have been formed and pulling the tool and tether ends back through the penetrations.

5. A method as in claim 1, wherein a single tether having the first end and the second end is advanced through the penetrations and joined to form the tether structure.

6. A method as in claim 1, wherein separate first and second tethers are advanced through the first and second penetrations, further comprising joining at least third and fourth ends of the tethers to form the tether structure.

7. A method as in claim 1, wherein separate first and second tethers are advanced through the first and second penetrations from opposite sides of the midline, further comprising joining the back end of the first tether to the front end of the second tether and the back end of the second tether to front end of the first tether.

8. A method as in claim 1, further comprising joining additional components as part of the tether structure.

9. A method as in claim 1, wherein the additional components are selected from compliance members, extension members, compression members, tension members, attachment buckles, and adjustment members.

10. A method as in claim 1, wherein the penetrations are expanded before passing of the tether.

11. A method as in claim 1, wherein a tool separate from the piercing tool is used to advance the tether.

12. A method for constraining spinous processes to elastically limit flexion of a spinal segment, said method comprising:
piercing an interspinous ligament with a tool to form a first penetration above an upper side of a first spinous process;
advancing a first end of a tether through the first penetration;
and attaching at least first and second ends of a tether to a lower spinous process or sacrum to form a tether structure wherein the tether structure elastically couples an upper spinous process and a lower spinous process or sacrum.

13. A method as in claim 1, further comprising adjusting the tether structure.

14. A method as in claim 13, wherein adjusting consists of changing an elastic component in the tether structure to increase or decrease the stiffness of the band.

15. A method as in claim 13, wherein adjusting consists of changing the effective length of the tether structure.

16. A method as in claim 15, wherein such changes in effective length are effected by increasing or decreasing the portion of a tether that is part of the continuous structure.

17. A method as in claim 15, further comprising engaging at least one tool with the continuous structure to effect such changes in effective length.

18. A method as in claim 15, wherein adjusting consists of changing the effective length of the continuous structure to achieve a targeted effective length.

19. A method as in claim 18, wherein the targeted effective length is determined by measuring landmarks on images of a patient's spine.

20. A method as in claim 19, wherein the landmarks are points on the patient's spinous processes.

21. A method as in claim 18, wherein the targeted effective length is determined by measuring the force resisting segmental flexion.

22. A method as in claim 15, wherein adjusting consists of changing the effective length of the continuous structure to achieve a targeted tension in the continuous structure.

23. A method as in claim 22, wherein the targeted tension in the continuous structure is determined by measuring the force resisting segmental flexion.

24. A method as in claim 15, further comprising a second adjustment step performed subsequent to the surgery.

25. A method as in claim 24, wherein the second adjustment step is performed by percutaneously engaging a tool with the tether and changing the perimeter of the continuous structure.

26. A method as in claim 24, wherein the second adjustment step is performed by transcutaneously communicating with a receiver on the device in order to activate components which adjust the continuous structure.

27. A method as in claim 13, further comprising holding the spinous processes at a fixed distance from each other while adjusting the tether structure.

28. A method as in claim 27, further comprising engaging a tool with the superior and inferior spinous processes at the targeted spinal segment to hold the spinous processes at a fixed distance from each other while the tether structure is adjusted around them.

29. A method as in claim 1, further comprising engaging a positioning guide against a preselected surface of the spinal segment; and positioning the tether along an anterior posterior axis defined by the guide.

30. A method as in claim 29, wherein positioning comprises engaging the tether against a feature of the guide when the guide is engaged against the surface of the spinous process.

31. A system for constraining adjacent spinous processes, said system comprising:
a tether; and
a piercing tool having a tissue-penetrating distal tip and an anchor for releasably attaching an end of the tether;
wherein the piercing tool is adapted to be advanced in an anterior direction from the skin toward the interspinous ligament and then towards the opposite side so that the tissue-penetrating tip can be pierced through the ligament to push or pull the attached tether through the resulting penetration.

32. A system as in claim 31, wherein the piercing tool is an elongate shaft having a C-shaped arm disposed in a perpendicular plane at a distal end thereof.

33. A system as in claim 31, wherein the piercing tool is marked so that the distance between the upper and lower piercing of the interspinous ligament can be determined.

34. A system as in claim 31, wherein the piercing tool is constructed to allow for both interspinous ligament piercings to be done simultaneously.

35. A system as in claim 31, further comprising an adjustment tool for adjusting the band.

36. A system as in claim 35, wherein engaging the adjustment tool with the tether and using the tool changes the effective length of the tether.

37. A system as in claim 35, wherein the adjustment tool consists of a screwdriver.

38. A system as in claim 37, wherein the head of the screwdriver is oriented at an angle to the shaft of the screwdriver.

39. A system as in claim 35, further comprising a stabilizing tool for holding the tether while the adjustment tool changes the effective length of the tether.

40. A system as in claim 35, further comprising a stabilizing tool for holding the spinous processes at a preset distance.
